(19) United States
(12) Patent Application Publication
(10) Pub. No.: US 2006/0002831 A1
(43) Pub. Date: Jan. 5, 2006

(54) Reactor system with several reactor units in parallel

(76) Inventor: Hans Georg Leffer, Saarbrucken (DE)

Correspondence Address: Jennifer D Adamson;Shell Oil Company, Intellectual Property, P O Box 2463, Houston, TX, 77252-2463, US

(21) Appl. No.: 10/521,161

(22) PCT Filed: Jun. 20, 2003
(86) PCT No.: PCT/EP03/06456

§ 371 Date: Jan. 3, 2005

(30) Foreign Application Priority Data
Jul. 4, 2002 (EP) 02254697.2

(51) Int. Cl. B01J 10/00; C07C 27/26

(52) U.S. Cl. 422/188; 518/726

(57) ABSTRACT

The invention concerns a reactor system suitable for carrying out chemical reactions, having one or more common reactant feed lines, two or more single unit operated reactor sections and one or more common product discharge lines. The reactor system is especially suitable for the production of hydrocarbons from synthesis gas over a catalyst.

The present invention relates to a reactor system suitable for carrying out chemical reactions, the system comprising two or more single unit operated reactor sections. More specifically, the invention concerns the catalytic conversion of synthesis gas into long chain hydrocarbons in a reactor system comprising a multitude of multitubular fixed bed reactors sections.

Much attention has been given in the past and is still given at the present moment to the scale-up of chemical processes, which in most cases results in the scale-up of chemical reactors. Usually it is more efficient (economical) to use one large scale reactor than a multitude of independently operated smaller reactors.

An important requirement in the scale-up of reactors, especially chemical reactors, is that a large, commercial reactor should operate in a predictable fashion, which could be a factor of 10,000 larger or even more than laboratory and development reactors. It is important that the reactor operates within a safe set of conditions with a predictable output and quality at a predictable cost. Changing the scale of a reaction alters the heat removal and mixing characteristics of the reaction zone, which may result in differences in temperature and concentration profiles. This may than result in amended chemistry, thus influencing productivity, selectivity, catalyst deactivation etc. of the reactor. This means that the performance of a large reactor is difficult to predict on the basis of the performance a small reactor. Thus, extensive scale-up tests, reactor modelling and basic reactor study are usually required for the scale-up of new and/or existing chemical reactors, for new as well for existing chemical reactions.

Quite often a natural maximum appears to exist in the scaling-up process of chemical reactors. Further scale-up would introduce too many uncertainties in the extrapolations based on the developed reactor models and/or is simply not practical.

The present invention tries to find another way for the scale-up or the further scale-up chemical reactors. Rather than simply increasing the size of an existing reactor (including adaptation of the reactor internals, catalyst beds, mixing internals, cooling system, feed lines/feed distribution, product withdrawal etc.), either in diameter and/or height, two or more reactors of a certain, preferably identical, size are combined and operated as one single unit. The common feed lines, i.e. gas and/or liquid reactor system feed lines, are divided into as many equivalent streams as there are reactors and introduced into the different equivalent reactors. Cooling and/or heating systems are shared between the reactors. There will be one or more common product discharge lines. The reactors are operated as one single unit. The control of the reactant feed to the reactor system is carried out by means of managing the feed flow (amount, temperature, composition, pressure etc.) in the one or more common reactant feed lines. There is in the single unit operation no individual control of each reactor sections. The control of the total product flow of the reactor system in the single unit operated is done by managing the product flow in the one or more common product discharge lines. In this type of operation there is no individual control of the product discharge of each reactor section. Thus, it is not possible to take one or more reactors out of operation. Only the complete reactor system can be taken out of operation. It is not possible to influence the condition in one of the reactors in a different way as in one of the other reactors. Turning of one of the reactant feed lines will result in the fact that none of the reactors will receive the reactant feed stream any longer. Closing one of the product discharge lines will result in the fact that none of the reactors will be able any more to discharge its products. Independent heating or cooling of the reactor sections is not possible. Reactor control will be based on information obtained from all reactors present. A runaway in one of the reactors cannot be solved by closing down the reactor involved. It has to result in the shut down of the complete system. Feed stream control is carried out by control of the common feed gas/liquid reactant feed lines.

The present invention therefore relates to a reactor system suitable for carrying out chemical reactions, comprising one or more common reactant feed lines, two or more single unit operated reactor sections and one or more common product discharge lines.

A main advantages of the present reactor system is the fact that scaling-up becomes easier. For instance, when a reactor of a certain size has proven to perform its tasks well, there is no need for a further scale-up of the reactor. Combining a multitude of similar reactors and operating it as one single unit with common reactant feed lines and common product discharge lines will result in the desired scale-up. Or, in the case that a certain (large) scale-up for a specific reactor is required, the scale-up can be limited by using e.g. three or four reactor sections operated as a single unit. The scale-up in than reduced by a factor three or four. Further advantages are the lower weight of the reactor, making transport/handling/lifting easier. It will be appreciated that the size of a reactor may be restricted by workshop limitations, road limitations, bridge limitations, lifting equipment limitations etc. The smaller size of the reactor may result in the fact that more companies are able to produce the reactor. Also simultaneous production by one or more vendors will be possible. As the reactor system is single-unit operated, there is no additional workforce needed to operate the unit from the control room. From a process control point of view there is no difference between one large reactor and the reactor system of the present invention: the reactor system of the present invention is operated in the same way as one single large reactor. In general, the heat-up/cool-down rates for the reactor system according to the present invention will be faster than for one large single reactor. Some additional maintenance may be required, while also a somewhat larger plot space may be required. However, these small disadvantages are clearly set off by the advantages. In addition, maintenance within the reactor may be done quicker, as work will be divided over several places.

The above described reactor system is especially useful for strongly exothermic reactions. An example is the conversion of synthesis gas, a mixture of carbon monoxide and hydrogen, into methanol or hydrocarbons. As these conversions are highly exothermic, it will be appreciated that extensive cooling is necessary. This results in an relatively high amount of cooling internals inside the reactor, resulting in a reactor which reaches relatively quickly its natural limits in scaling-up. Another example is the oxidation of (lower) olefins, e.g. the catalytical conversion of ethylene into ethylene-oxide in a multitubular fixed bed reactor. The reactor system is also suitable for biochemical reactions.

The reactor system according to the present invention suitably comprises between two and twenty single units operated reactor section, preferably between three and eight single unit operated reactor sections, more preferably comprises four sections. Usually a reactor section will comprise a more or less conventional reactor, i.e. an elongated cylindrical reactor, which, when in use, will be a vertical reactor. Suitable reactor sections are the well known chemical reactors as tank reactors, (multi) tubular reactors, tower reactors, fluidised bed reactors and slurry phase reactors. See for instance Perry's Chemical Engineers' Handbook (MgGraw-Hill Book Company, 6th edition, 4-24-4-27) and Chemical Reactor Design and Operation (Westerterp, Van Swaaij an Beenackers, John Wiley & Sons, 1984). It is also possible that the reactor sections are located in one large reactor. This will overcome a number of the problems related to scaling-up, however, some advantages as described above may disappear. Preferably, all reactor sections have the same size. However, this is not essential, and different sizes of reactors may be used. It will be appreciated that in that case measures have to be taken that the feed is distributed in the desired ratio over the reactors. Also cooling/heating systems may need adaptation. The single unit operated reactor sections will be operated in parallel. The reactor system does not comprise reactor sections which are operated in series. Preferably each reactor section is a separated, individual chemical reactor, suitably comprising a shell (or vessel) and one or more reaction zone.

In most cases each reactor section will comprises one or more catalyst beds. Also slurry reactors may be used. In view of the large heat generation in hydrocarbon synthesis from syngas, slurry reactors may have advantages over fixed bed reactors in terms of heat transfer. On the other hand major technical issues associated with slurry reactors include hydrodynamics and solids management. In a preferred embodiment the reactor sections comprise a multitubular fixed bed catalyst arrangement. The tubes are filled with catalyst particles, the tubes are surrounded by cooling medium, especially a mixture of water and steam. Thus, the reactor sections each comprise an indirect heat exchange system, which heat exchange systems are jointly operated. Preferably the well known thermosiphon system is to be used.

Depending on the chemical reaction to be carried out, gaseous and/or liquid feeds are to be introduced in the reactor. All possible reactor flow regimes may be used, i.e. up-flow and/or downflow, cocurrent and/or countercurrent. Also gas and/or liquid recycles may be used. In the case of the synthesis of hydrocarbons, one common gas reactant feed line will introduce the syngas into the reactor system. This feed is split up in as many streams as are necessary for the number of attached reactor sections, and fed to the different reactor sections. In the case that gas and liquid have to be introduced in the reactor sections, there is preferably a separated gas feed line and a separated liquid feed line. It is recommended that reactors of the same type are used in the system according to the invention, preferably of the same size. In the case of heterogeneous catalytic reactions preferably the same catalyst is used in all reactor sections, although this is not essential.

Depending on the chemical reaction to be carried out, gas and/or liquid have to be discharged from the reactor. In some cases slurry, e.g. a mixture of catalyst and liquid, has to be discharged from the reactor. When gas and liquid have to be discharged from the reactor, this may be done by means of a single discharge line, but preferably the reactor system comprises one common gas product discharge line and one common liquid reactant discharge line. The above described reactor system may comprise a gas and/or liquid recycle line between the common product discharge line and the common reactant feed line.

Suitably the reactor sections in the reactor system of the present invention are identical. Size, catalyst, design, cooling capacity etc. are similar. This is the preferred option as reactor manufacture in that case is a simple duplication process. However, identical reactor sections are not essential. Different sizes may be used, as well as different types of catalyst may be used. It will be appreciated that measures have to be taken that a correct feed distribution over the reactors has to be made, depending on the differences in design, catalyst etc. Also the cooling capacity may be different from one reactor to another, resulting in different conditions in the reactor sections of one reactor system. It should be taken into account, that once different conditions are created in one or more reactor section of the system according to the invention, there are no possibilities to change the conditions in one or more of the reactors, as the system is operated as one single unit.

The hydrocarbon synthesis as mentioned above may be any suitable hydrocarbon synthesis step known to the man skilled in the art, but is preferably a Fischer Tropsch reaction. The synthesis gas to be used for the hydrocarbon synthesis reaction, especially the Fischer Tropsch reaction, is made from a hydrocarbonaceous feed, especially by partial oxidation, catalytic partial oxidation and/or steam/methane reforming. In a suitable embodiment an autothermal reformer is used or a process in which the hydrocarbonaceous feed is introduced into a reforming zone, followed by partial oxidation of the product thus obtained, which partial oxidation product is used for heating the reforming zone. The hydrocarbonaceous feed is suitably methane, natural gas, associated gas or a mixture of C₁₋₄ hydrocarbons, especially natural gas.

To adjust the H₂/CO ratio in the syngas, carbon dioxide and/or steam may be introduced into the partial oxidation process and/or reforming process. The H₂/CO ratio of the syngas is suitably between 1.3 and 2.3, preferably between 1.6 and 2.1. If desired, (small) additional amounts of hydrogen may be made by steam methane reforming, preferably in combination with the water shift reaction. The additional hydrogen may also be used in other processes, e.g. hydrocracking.

The synthesis gas obtained in the way as described above, usually having a temperature between 900 and 1400° C., is cooled to a temperature between 100 and 500° C., suitably between 150 and 450° C., preferably between 300 and 400° C., preferably under the simultaneous generation of power, e.g. in the form of steam. Further cooling to temperatures between 40 and 130° C., preferably between 50 and 100° C., is done in a conventional heat exchanger, especially a tubular heat exchanger.

The purified gaseous mixture, comprising predominantly hydrogen and carbon monoxide, is contacted with a suitable catalyst in the catalytic conversion stage, in which the normally liquid hydrocarbons are formed.

The catalysts used for the catalytic conversion of the mixture comprising hydrogen and carbon monoxide into hydrocarbons are known in the art and are usually referred to as Fischer-Tropsch catalysts. Catalysts for use in this process frequently comprise, as the catalytically active component, a metal from Group VIII of the Periodic Table of Elements. Particular catalytically active metals include ruthenium, iron, cobalt and nickel. Cobalt is a preferred catalytically active metal.

The catalytically active metal is preferably supported on a porous carrier. The porous carrier may be selected from any of the suitable refractory metal oxides or silicates or combinations thereof known in the art. Particular examples of preferred porous carriers include silica, alumina, titania, zirconia, ceria, gallia and mixtures thereof, especially silica, alumina and titania.

The amount of catalytically active metal on the carrier is preferably in the range of from 3 to 300 pbw per 100 pbw of carrier material, more preferably from 10 to 80 pbw, especially from 20 to 60 pbw.

If desired, the catalyst may also comprise one or more metals or metal oxides as promoters. Suitable metal oxide promoters may be selected from Groups IIA, IIIB, IVB, VB and VIB of the Periodic Table of Elements, or the actinides and lanthanides. In particular, oxides of magnesium, calcium, strontium, barium, scandium, yttrium, lanthanum, cerium, titanium, zirconium, hafnium, thorium, uranium, vanadium, chromium and manganese are very suitable promoters. Particularly preferred metal oxide promoters for the catalyst used to prepare the waxes for use in the present invention are manganese and zirconium oxide. Suitable metal promoters may be selected from Groups VIIB or VIII of the Periodic Table. Rhenium and Group VIII noble metals are particularly suitable, with platinum and palladium being especially preferred. The amount of promoter present in the catalyst is suitably in the range of from 0.01 to 100 pbw, preferably 0.1 to 40, more preferably 1 to 20 pbw, per 100 pbw of carrier. The most preferred promoters are selected from vanadium, manganese, rhenium, zirconium and platinum.

The catalytically active metal and the promoter, if present, may be deposited on the carrier material by any suitable treatment, such as impregnation, kneading and extrusion. After deposition of the metal and, if appropriate, the promoter on the carrier material, the loaded carrier is typically subjected to calcination. The effect of the calcination treatment is to remove crystal water, to decompose volatile decomposition products and to convert organic and inorganic compounds to their respective oxides. After calcination, the resulting catalyst may be activated by contacting the catalyst with hydrogen or a hydrogen-containing gas, typically at temperatures of about 200 to 350° C. Other processes for the preparation of Fischer Tropsch catalysts comprise kneading/mulling, often followed by extrusion, drying/calcination and activation.

The catalytic conversion process may be performed under conventional synthesis conditions known in the art. Typically, the catalytic conversion may be effected at a temperature in the range of from 150 to 300° C., preferably from 180 to 260° C. Typical total pressures for the catalytic conversion process are in the range of from 1 to 200 bar absolute, more preferably from 10 to 70 bar absolute. In the catalytic conversion process especially more than 75 wt % of C₅⁺, preferably more than 85 wt % C₅⁺ hydrocarbons are formed. Depending on the catalyst and the conversion conditions, the amount of heavy wax (C₂₀⁺) may be up to 60 wt %, sometimes up to 70 wt %, and sometimes even up till 85 wt %. Preferably a cobalt catalyst is used, a low H₂/CO ratio is used and a low temperature is used (190-230° C.). To avoid any coke formation, it is preferred to use an H₂/CO ratio of at least 0.3. It is especially preferred to carry out the Fischer Tropsch reaction under such conditions that the SF-alpha value, for the obtained products having at least 20 carbon atoms, is at least 0.925, preferably at least 0.935, more preferably at least 0.945, even more preferably at least 0.955.

Preferably, a Fischer-Tropsch catalyst is used, which yields substantial quantities of paraffins, more preferably substantially unbranched paraffins. A most suitable catalyst for this purpose is a cobalt-containing Fischer-Tropsch catalyst. Such catalysts are described in the literature, see e.g. AU 698392 and WO 99/34917.

The Fischer Tropsch process may be a slurry FT process or a fixed bed FT process, especially a multitubular fixed bed.

The present invention also relates to a process for the preparation of hydrocarbons by reaction of carbon monoxide and hydrogen in the presence of a catalyst at elevated temperature and pressure, in which a reactor system is used as described above. Further, the invention relates to the products made in the Fischer Tropsch process. The present invention also relates to the preparation of methanol and to methanol as prepared, as well as to a process for the catalytic conversion of ethane into ethylene oxide.

1. A reactor system suitable for carrying out chemical reactions comprising one or more common reactant feed lines fed into two or more single unit operated reactor sections having one or more common product discharge lines, wherein each reactor section comprises an individual reactor.

2. The reactor system of claim 1, comprising between 3 and 8 single unit operated reactor sections.

3. The reactor system of claim 1, in which each reactor section comprises one or more catalyst beds.

4. The reactor system of claim 1, in which each of the reactor sections comprises an indirect heat exchange system, which heat exchange systems are jointly operated.

5. The reactor system of claim 1 comprising one common gas reactant feed line.

6. The reactor system of claim 1 comprising one common gas product discharge line.

7. The reactor system of claim 1 comprising one common liquid reactant discharge line or which system comprises one common liquid product discharge line.

8. (canceled)

9. A process for the preparation of hydrocarbons by reaction of carbon monoxide and hydrogen in the presence of a catalyst at elevated temperature and pressure, wherein the process is performed in a reactor system comprising one or more common reactant feed lines fed into two or more single unit operated reactor sections having one or more common product discharge lines, wherein each reactor section comprises an individual reactor.

10. The reactor system of claim 1 comprising four single unit operated reactor sections.

11. The reactor system of claim 1, wherein each reactor section comprises a multitubular fixed bed catalyst arrangement.

12. The reactor system of claim 4, wherein the heat exchange system comprises a thermosiphon system.

13. The reactor system of claim 1 comprising one common liquid product discharge line.

14. The process of claim 9, wherein the catalyst comprises a cobalt catalyst.
